(19) United States
(12) Patent Application Publication
(10) Pub. No.: US 2004/0044055 A1
(43) Pub. Date: Mar. 4, 2004

(54) N-alkoxlyalkyl-substituted benzimidazoles and the use thereof as an agent against parastic protozoans

(76) Inventors: Folker Lieb, Leverkusen (DE); Albrecht Marhold, Leverkusen (DE); Torsten Neugebauer, Bad Honnef (DE); Gisela Greif, Remagen (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/398,295

(22) PCT Filed: Sep. 24, 2001
(86) PCT No.: PCT/EP01/11010

(30) Foreign Application Priority Data
Oct. 6, 2000 (DE) 100 49 468.4

(51) Int. Cl. C07D 235/22; A61K 31/4184

(52) U.S. Cl. 514/394; 548/310.1

(57) ABSTRACT

The present invention relates to novel N-alkoxyalkyl-substituted benzimidazoles, to their preparation and to their use as agents against parasitic protozoa.

The present invention relates to novel N-alkoxyalkyl-substituted benzimidazoles, to their preparation and to their use as agents against parasitic protozoa.

The present invention further relates to mixtures of these compounds with polyether antibiotics or synthetically prepared coccidiosis agents in compositions for the control of parasitic protozoa, in particular coccidia.

Substituted benzimidazoles and their use as insecticides, fungicides and herbicides have already been disclosed (EP-A 87 375, 152 360, 181 826, 239 508, 260 744, 266 984, U.S. Pat. Nos. 3,418,318, 3,472,865, 3,576,818, 3,728,994).

Halogenated benzimidazoles and their action as anthelmintics, coccidiostatics and pesticides have already been disclosed (German Offenlegungsschrift 2 047 369, German Offenlegungsschrift 4 237 617). Mixtures of nitro-substituted benzimidazoles and polyether antibiotics have been disclosed as coccidiosis agents (U.S. Pat. Nos. 5,331,003, 5,670,485). Mixtures of other substituted benzimidazole compounds and polyether antibiotics or synthetic coccidiosis agents are known (WO 96/38140, WO 00/04022).

Coccidiosis is a disorder which is caused by single-cell parasites (protozoa). It can cause great losses, in particular when raising poultry. In order to avoid these, the stocks are treated prophylactically with coccidiosis agents. Owing to the development of resistance to the agents employed, serious problems already occur shortly after introduction of the agents. By means of the use of chemically completely new coccidiosis agents, in particular combinations, it is possible, on the other hand, to control even polyresistant parasite strains.

Accordingly, there is still a need for novel active compounds and compositions having improved properties for controlling diseases caused by parasitic protozoa.

The present invention relates to novel benzimidazoles of the formula (I)

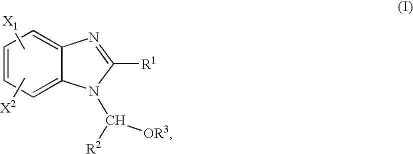

in which

R¹ represents fluoroalkyl,

R² represents hydrogen or alkyl,

R³ represents alkyl,

X¹ represents trifluoromethyl and

X² represents trifluoromethoxy,

which show excellent activity against coccidiosis.

The benzimidazoles of the formula (I)

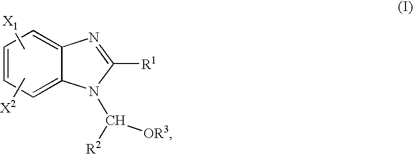

in which

R¹, R², R³, X¹ and X² have the meanings given above,

are prepared by reacting

1H-benzimidazoles of the formula (II)

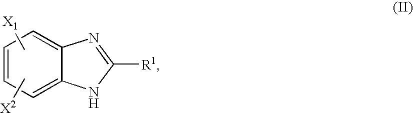

in which

R¹, X¹ and X² have the meaning given above

with an alkylating agent of the formula (III)

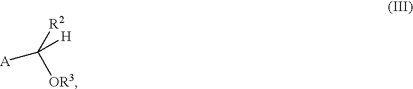

in which

A represents a suitable leaving group,

R² and R³ have the meaning given above, if appropriate in the presence of diluents and/or reaction auxiliaries.

If appropriate, the compounds of the formula (I) can be present as geometrical and/or optical isomers or regioisomers or isomer mixtures thereof in varying composition, depending on the nature and number of substituents. If appropriate, the isomers can be separated in a manner known per se, for example by crystallization or chromatographic methods. Both the pure isomers and the isomer mixtures are claimed according to the invention.

The compounds according to the invention can also be present as salts. In the context of the invention, preference is given to physiologically acceptable salts.

Physiologically acceptable salts may be salts of the compounds according to the invention with inorganic or organic acids. Preference is given to salts with inorganic acids such as, for example, hydrochloric acid, hydrobromic acid, phosphoric acid or sulphuric acid or to salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

The formula (I) provides a general definition of the substituted benzimidazoles according to the invention. Preference is given to compounds of the formula (I) in which

R¹ represents C₁-C₄-fluoroalkyl,

R² represents hydrogen or C₁-C₄-alkyl,

R³ represents C₁-C₈-alkyl,

X¹ represents trifluoromethyl and

X² represents trifluoromethoxy.

Particular preference is given to compounds of the formula (I) in which

R¹ represents CF₃, CHF₂, CH₂F,

R² represents hydrogen, methyl, ethyl, n-propyl or isopropyl,

R³ represents C₁-C₆-alkyl,

X¹ represents trifluoromethyl and

X² represents trifluoromethoxy.

Very particular preference is given to compounds of the formula (I) in which

R¹ represents —CF₃,

R² represents hydrogen,

R³ represents methyl, ethyl, propyl, iso-propyl, butyl, iso-butyl or sec-butyl,

X¹ represents trifluoromethyl and

X² represents trifluoromethoxy.

Alkyl generally represents a straight-chain or branched alkyl radical having preferably up to 8, particularly preferably up to 6, very particularly preferably up to 4, carbon atoms. Examples are: methyl, ethyl, n-propyl, iso-propyl, n-butyl, iso-butyl (=sec-butyl).

Fluoroalkyl generally represents a straight-chain or branched alkyl radical having preferably up to 6, particularly preferably up to 4, very particularly preferably up to 3, carbon atoms in which at least one, preferably more than one, particularly preferably all, hydrogens are replaced by fluorine. Examples are: trifluoromethyl, difluoromethyl, pentafluoroethyl, etc.

Using, for example, 5-trifluoromethoxy-2,6-bis-trifluoromethylbenzimidazole for carrying out the process according to the invention for preparing compounds of the formula (I), the course of the reaction of the preparation process can be represented by the following equation:

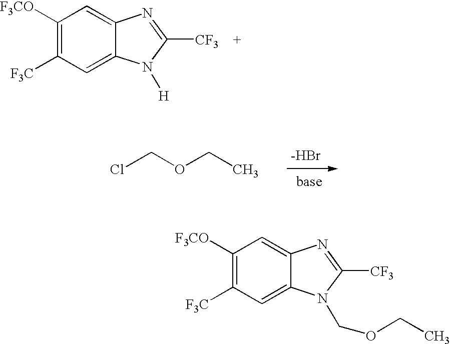

Formula (I) provides a general definition of the 1H-benzimidazoles needed as starting substances for carrying out the preparation process. In this formula (II), R¹ to R³ and X¹ and X² preferably represent those radicals which have already been mentioned as preferred for these substituents in connection with the description of the compounds of the formula (I) according to the invention.

The 1H-benzimidazoles of the formula (II) are known or can be obtained in analogy to known processes (cf., for example, J. Amer. Chem. Soc. 75, 1292 [1953] U.S. Pat. No. 3,576,818).

Formula (III) provides a general definition of the alkylating agents furthermore necessary as starting materials for carrying out the preparation process. In this formula (III), R² and R³ preferably represent those radicals which have already been mentioned as preferred for these substituents in connection with the description of the substances of the formula (I) according to the invention.

A represents a leaving radical customary in alkylating agents, preferably halogen, in particular chlorine, bromine or iodine or alkylsulphonyloxy, alkoxysulphonyloxy or arylsulphonyloxy, each of which is optionally substituted, such as, in particular, methanesulphonyloxy, trifluoromethanesulphonyloxy, methoxysulphonyloxy, ethoxysulphonyloxy or p-toluenesulphonyloxy.

The compounds of the formula (III) are generally known or can be obtained in analogy to known processes (cf., for example, J. Amer. Chem. Soc. 93, 5725-5731, Synthesis 1982, 942-944).

Suitable diluents for carrying out the preparation process are inert organic solvents. These in particular include aliphatic, alicyclic or aromatic, optionally halogenated hydrocarbons, such as, for example, benzine, benzene, toluene, xylene, chloro-benzene, dichlorobenzene, petroleum ether, hexane, cyclohexane, dichloromethane, chloroform or carbon tetrachloride; ethers, such as diethyl ether, diisopropyl ether, dioxane, tetrahydrofuran or ethylene glycol dimethyl or ethylene glycol diethyl ether; ketones, such as acetone, butanone or methyl isobutyl ketone; nitriles, such as acetonitrile, propionitrile or benzonitrile; amides, such as N,N-dimethylformamide, N,N-dimethylacetamide, N-methylformanilide, N-methylpyrrolidone or hexamethyl-phosphoramide; esters, such as methyl acetate or ethyl acetate, or bases such as pyridine.

The preparation process is preferably carried out in the presence of a suitable reaction auxiliary. Those suitable are all customary inorganic or organic bases. These include, for example, alkaline earth metal or alkali metal hydrides, hydroxides, amides, alcoholates, acetates, carbonates or hydrogen carbonates, such as, for example, sodium hydride, sodium amide, lithium diethylamide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium hydrogen carbonate, sodium hydrogen carbonate or ammonium carbonate, organolithium compounds, such as n-butyllithium, and also tertiary amines, such as trimethylamine, triethylamine, tributylamine, di-isopropyl-ethylamine, tetramethylguanidine, N,N-dimethylaniline, pyridine, piperidine, N-methylpiperidine, N,N-dimethylaminopyridine, diazabicyclooctane (DABCO), diazabicyclononene (DBN) or diazabicycloundecene (DBU).

The preparation process can optionally also be carried out in a two-phase system, such as, for example, water/toluene or water/dichloromethane, if appropriate in the presence of a suitable phase-transfer catalyst. Examples of such catalysts which may be mentioned are: tetrabutylammonium iodide, tetrabutylammonium bromide, tetrabutylammonium chloride, tributyl-methylphosphonium bromide, trimethyl-C₁₃/C₁₅-alkylammonium chloride, trimethyl-C₁₃/C₁₅-alkylammonium bromide, dibenzyl-dimethyl-ammonium methylsulphate, dimethyl-C₁₂/C₁₄-alkyl-benzyl-ammonium chloride, dimethyl-C₁₂/C₁₄-alkyl-benzylammonium bromide, tetrabutylammonium hydroxide, triethylbenzylammonium chloride, methyltrioctyl-ammonium chloride, trimethylbenzylammonium chloride, 15-crown-5, 18-crown-6 or tris-[2-(2-methoxyethoxy)-ethyl]-amine.

When carrying out the preparation process, the reaction temperatures can be varied within a relatively wide range. In general, the reaction is carried out at temperatures between −70° C. and +200° C., preferably at temperatures between 0° C. and 130° C.

The preparation process is customarily carried out under normal pressure. However, it is also possible to work at increased or reduced pressure.

To carry out the preparation process, in general 1.0 to 5.0 mol, preferably 1.0 to 2.5 mol, of alkylating agent of the formula (III) and, if appropriate, 0.01 to 5.0 mol, preferably 1.0 to 3.0 mol, of reaction auxiliary are employed per mole of 1H-benzimidazole of the formula (II).

The reaction is carried out and worked up and the reaction products are isolated according to known processes (cf. also the Preparation Examples).

The final products of the formula (I) are purified with the aid of customary processes, for example by column chromatography or by recrystallization.

Characterization is carried out with the aid of the melting point or in the case of non-crystallizing compounds—in particular in the case of regioisomer mixtures—with the aid of proton nuclear magnetic resonance spectroscopy (¹H-NMR).

The active compounds have favourable toxicity to warm-blooded animals and are suitable for the control of parasitic protozoa which occur in animal husbandry and animal breeding in the case of useful, breeding, zoo, laboratory and experimental animals and pets. At the same time, they are active against all or individual stages of development of the pests and also against resistant and normally sensitive strains. By means of the control of the parasitic protozoa, illness, cases of death and yield reductions (e.g. in the production of meat, milk, wool, hides, eggs, honey etc.) should be decreased, so that simpler and more economical animal husbandry is possible due to the use of the active compounds.

The parasitic protozoa include:

Mastigophora (Flagellata) such as, for example, Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example, Giardia lamblia, G. canis.

Sarcomastigophora (Rhizopoda) such as Entamoebidae, for example, Entamoeba histolytica, Hartmanellidae, for example, Acanthamoeba sp., Hartmanella sp.

Apicomplexa (Sporozoa) such as Eimeridae, for example, Eimeria acervulina, E. adenoides, E. alabahmensis, E. anatis, E. anseris, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Neospara caninum, Neospora hughesi; Cystisospora spec., Cryptosporidium spec. such as Toxoplasmadidae, for example, Toxoplasma gondii, such as Sarcocystidae, for example, Sarcocystis bovicanis, S. bovibominis, S. neurona, S. ovicanis, S. ovifelis, S. spec., S. suihominis such as Leucozoidae, for example, Leucozytozoon simondi, such as Plasmodiidae, for example, Plasmodium berghei, P. falciparum, P. malariae, P. ovale, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Furthermore Myxospora and Microspora, for example, Glugea spec. Nosema spec.

Furthermore Pneumocystis carinii, and also Ciliophora (Ciliata) such as, for example, Balantidium coli, Ichthiophthirius spec., Trichodina spec., Epistylis spec.

The compounds according to the invention are also active against protozoa which occur as parasites in insects. Those which may be mentioned are parasites of the strain Microsporida, in particular of the genus Nosema. Particular mention may be made of Nosema apis in the case of the honeybee.

The useful and breeding animals include mammals, such as, for example, cattle, horses, sheep, pigs, goats, camels, water buffalo, donkeys, rabbits, fallow deer, reindeer, fur-bearing animals such as, for example, mink, chinchilla, racoons, birds, such as, for example, hens, geese, turkeys, ducks, doves, bird species for keeping at home and in zoos. Useful and ornamental fish are furthermore included.

The laboratory and experimental animals include mice, rats, guinea pigs, golden hamsters, dogs and cats.

The pets include dogs and cats.

The fish include useful, breeding, aquarium and ornamental fish of all age levels, which live in fresh and salt water. The useful and ornamental fish include, for example, carp, eels, trout, whitefish, salmon, bream, roach, rudd, chub, sole, plaice, halibut, Japanese yellowtail (Seriola quinqueradiata), Japanese eel (Anguilla japonica), red seabream (Pagurus major), seabass (Dicentrarchus labrax), grey mullet (Mugilus cephalus), pompano, gilthead seabream (Sparus auratus), Tilapia ssp., Chichlidae species such as, for example, Plagioscion, Channel catfish. The compositions according to the invention are particularly suitable for the treatment of fry, e.g. carp of 2 to 4 cm body length. The compositions are also very highly suitable in eel feeding.

Administration can be carried out both prophylactically and therapeutically.

The administration of the active compounds is carried out directly or enterally, parenterally, dermally or nasally in the form of suitable preparations.

Enteral administration of the active compounds takes place, for example, possible to take orally in the form of powders, suppositories, tablets, capsules, pastes, drinks, granules, drenches, boli, medicated feed or drinking water. Dermal administration takes place, for example, in the form of dipping, spraying, bathing, washing, pouring on and spotting on, and dusting. Parenteral administration takes place, for example, in the form of injection (intramuscular, subcutaneous, intravenous, intraperitoneal) or by means of implants.

Suitable preparations are:

Solutions such as injection solutions, oral solutions, concentrates for oral administration after dilution, solutions for use on the skin or in body cavities, pour-on formulations, gels;

Emulsions and suspensions for oral or dermal administration and for injection; semi-solid preparations;

Formulations in which the active compound is incorporated in an ointment base or in an oil-in-water or water-in-oil emulsion base.

Solid preparations such as powders, premixes or concentrates, granules, pellets, tablets, boli, capsules; aerosols and inhalations, active compound-containing shaped articles.

Injection solutions are administered intravenously, intramuscularly and sub-cutaneously.

Injection solutions are prepared by dissolving the active compound in a suitable solvent and possibly adding additives such as solubilizers, acids, bases, buffer salts, antioxidants, preservatives. The solutions are sterile-filtered and buffered.

Solvents which may be mentioned are: physiologically tolerable solvents such as water, alcohols such as ethanol, butanol, benzyl alcohol, glycerol, hydrocarbons, propylene glycol, polyethylene glycols, N-methylpyrrolidone, and mixtures thereof.

If appropriate, the active compounds can also be dissolved in physiologically tolerable vegetable or synthetic oils which are suitable for injection.

Solubilizers which may be mentioned are: solvents which promote the dissolution of the active compound in the main solvent or prevent its precipitation. Examples are polyvinylpyrrolidone, polyethoxylated castor oil, polyethoxylated sorbitan ester.

Preservatives are: benzyl alcohol, trichlorobutanol, esters of p-hydroxybenzoic acid, n-butanol.

Oral solutions are administered directly. Concentrates are used orally after prior dilution to the use concentration. Oral solutions and concentrates are prepared as described above in connection with injection solutions, it being possible to dispense with sterile operation.

Solutions for use on the skin are spotted on, painted on, rubbed in, squirted or sprayed on or applied by dipping, bathing or washing. These solutions are prepared as described above in connection with the injection solutions.

It may be advantageous to add thickeners during preparation. Thickeners are: inorganic thickeners such as bentonites, colloidal silica, aluminium monostearate, organic thickeners such as cellulose derivatives, polyvinyl alcohols and their copolymers, acrylates and methacrylates.

Gels are applied to or painted onto the skin or introduced into body cavities. Gels are prepared by mixing solutions, which have been prepared as described in connection with the injection solutions, with sufficient thickener to form a clear composition with an ointment-like consistency. Thickeners employed are the thickeners indicated further above.

Pour-on formulations are poured or squirted onto limited areas of the skin, the active compound either penetrating the skin and acting systemically or being dispersed on the surface of the body.

Pour-on formulations are prepared by dissolving, suspending or emulsifying the active compound in suitable skin-tolerable solvents or solvent mixtures. If appropriate, further auxiliaries such as colorants, absorption-promoting substances, antioxidants, sunscreen agents and/or adherents are added.

Solvents which may be mentioned are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, aromatic and/or aliphatic hydrocarbons, vegetable or synthetic oils, DMF, dimethylacetamide, N-methylpyrrolidone, 2-dimethyl-4-oxy-methylene-1,3-dioxolane.

Colorants are all colorants approved for use on animals and which can be dissolved or suspended.

Absorption-promoting substances are, for example, DMSO, spreading oils such as isopropyl myristate, dipropylene glycol pelargonate, silicone oils, esters of fatty acids, triglycerides, fatty alcohols.

Antioxidants are sulphites or metabisulphites such as potassium metabisulphite, ascorbic acid, butylhydroxytoluene, butylhydroxyanisole, tocopherol.

Sunscreen agents are, for example, substances from the benzophenones or novantisolic acid class.

Adherents are, for example, cellulose derivatives, starch derivatives, polyacrylates, natural polymers such as alginates, gelatine.

Emulsions can be used orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, further auxiliaries such as colorants, absorption-promoting substances, preservatives, antioxidants, sunscreen agents and/or viscosity-increasing substances.

Hydrophobic phases (oils) which may be mentioned are: paraffin oils, silicone oils, natural vegetable oils such as sesame oil, almond oil, castor oil, synthetic triglycerides such as caprylic/capric acid biglyceride, triglyceride mixture with vegetable fatty acids of chain length C₈₋₁₂ or other specially selected natural fatty acids, partial glyceride mixtures of saturated or unsaturated fatty acids possibly also containing hydroxyl groups, mono- and diglycerides of the C₈/C₁₀ fatty acids.

Esters of fatty acids such as ethyl stearate, di-n-butyryl adipate, hexyl laurate, dipropylene glycol pelargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleates, decyl oleates, ethyl oleate, ethyl lactates, waxy fatty acid esters such as dibutyl phthalate, diisopropyl adipate, ester mixtures related to the latter, inter alia fatty alcohols such as isotridecyl alcohol, 2-octyldodecanol, cetylstearyl alcohol, oleyl alcohol.

Fatty acids such as, for example, oleic acid and its mixtures.

Hydrophilic phases which may be mentioned are:

water, alcohols such as, for example, propylene glycol, glycerol, sorbitol and their mixtures.

Emulsifiers which may be mentioned are:

nonionic surfactants, e.g. polyethoxylated castor oil, polyethoxylated sorbitan monooleate, sorbitan monostearate, glycerol monostearate, polyoxyethyl stearate, alkylphenyl polyglycol ethers;

ampholytic surfactants such as di-Na N-lauryl-β-iminodipropionate or lecithin;

anionic surfactants, such as Na laurylsulphate, fatty alcohol ether sulphates, mono/dialkyl polyglycol ether orthophosphate monoethanolamine salt;

cationic surfactants such as cetyltrimethylammonium chloride.

Further auxiliaries which may be mentioned are:

viscosity-increasing and emulsion-stabilizing substances such as carboxymethyl-cellulose, methylcellulose and other cellulose and starch derivatives, polyacrylates, alginates, gelatine, gum arabic, polyvinylpyrrolidone, polyvinyl alcohol, copolymers of methyl vinyl ether and maleic anhydride, polyethylene glycols, waxes, colloidal silica or mixtures of the substances mentioned.

Suspensions can be used orally, dermally or as an injection. They are prepared by suspending the active compound in a suspending agent, if appropriate with addition of further auxiliaries such as wetting agents, colorants, absorption-promoting substances, preservatives, antioxidants, sunscreen agents.

Suspending agents which may be mentioned are all homogeneous solvents and solvent mixtures.

Wetting agents (dispersing agents) which may be mentioned are the surfactants indicated further above.

Further auxiliaries which may be mentioned are those indicated further above.

Semi-solid preparations can be administered orally or dermally. They differ from the suspensions and emulsions described above only by their higher viscosity.

To prepare solid preparations, the active compound is brought into the desired form with suitable excipients, if appropriate with addition of auxiliaries.

Excipients which may be mentioned are all physiologically tolerable solid inert substances. Those which are used are inorganic and organic substances. Inorganic substances are, for example, sodium chloride, carbonates such as calcium carbonate, hydrogen carbonates, aluminium oxides, silicas, argillaceous earths, precipitated or colloidal silica, phosphates.

Organic substances are, for example, sugar, cellulose, foodstuffs and feedstuffs such as powdered milk, animal meals, cereal meals and shreds, starches.

Auxiliaries are preservatives, antioxidants and colorants which have already been mentioned further above.

Further suitable auxiliaries are lubricants and glidants such as, for example, magnesium stearate, stearic acid, talc, bentonites, disintegration-promoting substances such as starch or crosslinked polyvinylpyrrolidone, binding agents such as, for example, starch, gelatine or linear polyvinylpyrrolidone and also dry binding agents such as microcrystalline cellulose.

The active compounds can be present in the preparations even as a mixture with synergists or with other active compounds.

Particular emphasis may be given to mixtures of the compounds according to the invention with a polyether antibiotic or a synthetic coccidiosis agent.

Synthetic coccidiosis agents or polyether antibiotics which may preferably be mentioned for use in the mixtures according to the invention are:

amprolium, in some cases in combination with folic acid antagonists

robenidine

toltrazuril

monensin

salinomycin

maduramicin

lasalocid

narasin

semduramicin.

From this list preference is given to monensin, salinomycin and maduramicin. Particular emphasis may be given to the mixture with maduramicin.

Ready-to-use preparations contain the active compounds in concentrations of 10 ppm to 20 per cent by weight, preferably from 0.1 to 10 per cent by weight.

Preparations which are diluted before use contain the active compound in concentrations of from 0.5 to 90 per cent by weight, preferably from 1 to 50 per cent by weight.

In general, it has proved advantageous to administer amounts from approximately 0.5 to approximately 50 mg, preferably 1 to 20 mg, of active compound per kg of body weight per day to achieve effective results.

In the mixture with other coccidiosis agents or polyether antibiotics, the active compounds according to the invention are in the ratio 1 to 0.1-10 to 1 to 1-10. The ratio 1 to 5 is preferred.

The active compounds can also be administered to the animals together with the feed or drinking water.

Feedstuffs and foodstuffs contain 0.01 to 250 ppm, preferably 0.5 to 100 ppm, of the active compound in combination with a suitable edible material.

Such a feedstuff and foodstuff can be used both for curative purposes and for prophylactic purposes.

Such a feedstuff or foodstuff is prepared by mixing a concentrate or a premix which contains 0.5 to 30%, preferably 1 to 20%, by weight of an active compound as a mixture with an edible organic or inorganic carrier with customary feedstuffs. Edible carriers are, for example, maize flour or maize and soya bean flour or mineral salts, which preferably contain a small amount of an edible dust prevention oil, e.g. maize oil or soya oil. The premix obtained in this way can then be added to the complete feedstuff before feeding it to the animals.

By way of example, use in coccidiosis may be mentioned:

For the healing and prophylaxis, for example, of coccidiosis in poultry, in particular in hens, ducks, geese and turkeys, 0.1 to 100 ppm, preferably 0.5 to 100 ppm, of an active compound are mixed with a suitable edible material, e.g. a nutritious feedstuff. If desired, these amounts can be increased, particularly if the active compound is well tolerated by the recipient. Correspondingly, administration can be carried out via the drinking water.

For the treatment of individual animals, e.g. in the case of the treatment of coccidiosis in mammals or of toxoplasmosis, amounts of active compound of 0.5 to 100 mg/kg of body weight are preferably administered daily in order to achieve the desired results. In spite of this, it may occasionally be necessary to depart from the amounts mentioned, in particular depending on the body weight of the experimental animal or on the type of administration method, but also because of the animal genus and its individual reaction to the active compound or the nature of the formulation and the time or the interval at which it is administered. Thus in certain cases it may be sufficient to manage with less than the abovementioned minimum amount, while in other cases the upper limit mentioned must be exceeded. When administering relatively large amounts, it may be advisable to divide these into several individual administrations during the course of the day.

The efficacy of the compounds according to the invention can be confirmed, for example, in cage experiments with the following experimental arrangement, in which the animals are treated with the respective individual components and with the mixtures of the individual components.

An active compound-containing feed is prepared such that the required amount of active compound is basically mixed with a nutritionally balanced animal feed, e.g. with the chick feed indicated below.

If a concentrate or a premix is to be prepared, which is finally to be diluted in the feed to the values mentioned in the experiment, in general approximately 1 to 30%, preferably approximately 10 to 20%, by weight of active compound are mixed with an edible organic or inorganic carrier, e.g. maize and soya meal or mineral salts which contain a small amount of an edible dedusting oil, e.g. maize oil or soya bean oil. The premix thus obtained can then be added to the complete poultry feed before administration.

A suitable example of the use of the substances according to the invention in the poultry feed is the following composition.

| | |
|---|---|
| 52.00% | of feed cereal shreds, that is: 40% maize, 12% wheat |
| 17.00% | of soya shreds extr. |
| 5.00% | of maize gluten feed |
| 5.00% | of wheat feed meal |
| 3.00% | of fish meal |
| 3.00% | of mineral mixture |
| 3.00% | of alfalfa meal |
| 2.50% | of vitamin premix |
| 2.00% | of wheat germs, comminuted |
| 2.00% | of soya oil |
| 2.00% | of meat and bone meal |
| 1.50% | of whey powder |
| 1.00% | of molasses |
| 1.00% | of brewer's yeast, bound to brewer's gains |
| 100.00% | |

Such a feed contains 18% raw protein, 5% raw fibre, 1% Ca, 0.7% P and, per kg, 1200 I.U. of vitamin A, 1200 I.U. of vitamin D3, 10 mg of vitamin E, 20 mg of zinc bacitracin.

The active compound is added to this feed in amounts of, for example, from 1 to 20 ppm (w/w). Suitable dosages of active compound are, for example, 1 ppm; 2.5 ppm; 5 ppm (in each case stated as parts by weight “(w/w)”).

Preparation Examples Compounds of the Formula (I)

### EXAMPLE 1

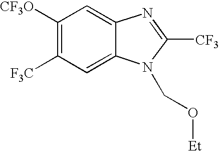

0.85 g (2.5 mmol) of 5-trifluoromethoxy-2,6-bis-trifluoromethyl-benzimidazole is initially charged into 25 ml of abs. methylene chloride and 0.45 ml (0.32 g) (3.15 mmol) of triethylamine is added at 20° C. At 20° C., 0.30 g (3.15 mmol) of chloromethyl ethyl ether in 2.5 ml of abs. methylene chloride is then added dropwise, and the mixture is refluxed for 24 h. The organic phase is washed three times with water and with aqueous sodium chloride solution, dried over sodium sulphate and concentrated. The residue is chromatographed over silica gel (35-70 μm) using cyclohexane/ethyl acetate (5:1).

Yield: 0.54 g (54% of theory), oil. R_{F}=0.47 [TLC: cyclohexane/ethyl acetate (5:1)], mixture of regioisomers (ratio 1:1).

### EXAMPLE 2

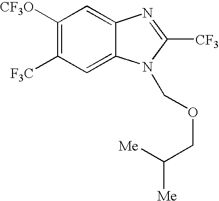

1.7 g (5 mmol) of 5-trifluoromethoxy-2,6-bis-trifluoromethyl-benzimidazole are initially charged in 50 ml of abs. methylene chloride and 0.9 ml (0.64 g) (6.3 mmol) of triethylamine are added at 20° C. At 20° C., 0.65 g (6.3 mmol) of chloromethyl 2-methylisopropyl ether in 5 ml of abs. methylene chloride are added dropwise and the mixture is refluxed for 16 h. The organic phase is washed twice with water and with aqueous sodium chloride solution, dried over sodium sulphate and concentrated. The residue is chromatographed over silica gel (35-70 μm) using cyclohexane/ethyl acetate (10:1).

Yield: 1.0 g (47% of theory), oil. R_{F}=0.48 [TLC: cyclohexane/ethyl acetate (5:1)], mixture of regioisomers (ratio 1:1).

### EXAMPLE3

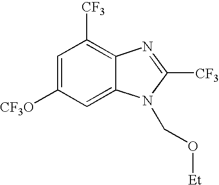

0.68 g (2 mmol) of 6-trifluoromethoxy-2,4-bis-trifluoromethyl-benzimidazole is initially charged in 20 ml of methylene chloride and 0.35 ml (0.25 g) (2.5 mmol) of triethylamine is added at 20° C. At 20° C., 0.19 g (2.5 mmol) of chloromethyl ethyl ether in 2.5 ml of abs. methylene chloride is then added dropwise and the mixture is refluxed for 16 h. The organic phase is washed three times with water and with aqueous sodium chloride solution, dried over sodium sulphate and concentrated. The residue is chromatographed over silica gel (35-70 μm) using cyclohexane/ethyl acetate (5:1).

Yield: 0.63 g (76% of theory), oil. R_{F}=0.5 [TLC: cyclohexane/ethyl acetate (5:1)].

Preparation of the Starting Materials for Examples 1 and 2

### Example a

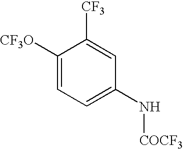

At room temperature, 122 g (0.5 mol) of 4-trifluoromethoxy-3-trifluoromethylaniline are initially charged in 500 ml of toluene and 5 ml of pyridine, 112 g (1.1 mol) of trifluoroacetic acid are added dropwise and the mixture is heated at reflux for 3 h using a water separator. The mixture is concentrated, the residue is mixed with water and extracted with methylene chloride and the organic phase is dried using sodium sulphate. The organic phase is concentrated and distilled under reduced pressure.

Yield: 109 g (64% of theory), b.p._{0.1}: 120-125° C., GC: 95.8%.

### Example b

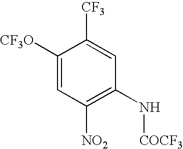

With cooling, 110 g (0.32 mol) of N-(4-trifluoromethoxy-3-trifluoromethyl-phenyl)-trifluoroacetamide are dissolved in 180 ml of conc. sulphuric acid. With ice cooling, a mixture of 180 ml of conc. sulphuric acid and 30 ml of conc. nitric acid is slowly added at 5° C. over a period of 5 h. The mixture is then poured onto ice and extracted with methylene chloride and the organic phase is washed with water until neutral and dried over sodium sulphate. The organic phase is concentrated and dried under reduced pressure. The crude product is reacted further.

Yield: 95 g (77% of theory), GC: >99%.

### Example c

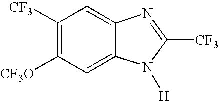

In an autoclave, 35 g (0.09 mol) of N-(2-nitro-4-trifluoromethoxy-5-trifluoromethyl-phenyl)-trifluoroacetamide are hydrogenated at a hydrogen pressure of 100 bar for 22 h using 8 g of Raney nickel in 200 ml of methanol. The Raney nickel is then filtered off and washed with methanol and the mother liquor is concentrated. The residue is admixed with methylene chloride, washed twice with a little water and dried over sodium sulphate. The residue still contains N-(2-amino4-trifluoromethoxy-5-trifluoromethyl-phenyl)-trifluoroacetamide (GC). For this reason, the organic phase is treated with 150 ml of trifluoroacetic acid and 20 ml of trifluoroacetic anhydride and heated under reflux for 14 h. The mixture is cooled and adjusted to pH 8 using 200 ml of 20% strength sodium hydroxide solution and then 50 ml of conc. sodium hydroxide solution. The mixture is extracted with methylene chloride and the organic phase is washed with water until neutral, dried over sodium sulphate and concentrated.

Yield: 10 g (32% of theory), GC: 64%.

Preparation of the Starting Material for Example 3

### Example d

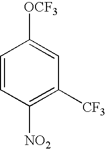

With cooling, 115 g (0.5 mol) of 3-trifluoromethylbenzotrifluoride are dissolved in 180 ml of conc. sulphuric acid. With ice cooling, a mixture of 180 ml of conc. sulphuric acid and 30 ml of conc. nitric acid is slowly added dropwise at 5° C. over a period of 5 h, the mixture is then poured onto ice and extracted with t-butyl methyl ether and the organic phase is washed with water until neutral and dried over sodium sulphate. The organic phase is concentrated and dried under reduced pressure.

Yield: 90 g (65% of theory), GC: >98%.

### Example e

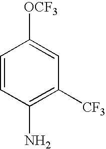

In an autoclave, 30 g (0.19 mol) of 4-trifluoromethoxy-2-trifluoromethyl-nitrobenzene are hydrogenated at a hydrogen pressure of 100 bar using 2 g of Raney nickel in 200 ml of methanol. The Raney nickel is then filtered off and washed with methanol and the organic phases are concentrated. The residue is mixed with methylene chloride, washed twice with a little water and dried over sodium sulphate.

Yield: 23.7 g (89% of theory), GC:>96%.

### Example f

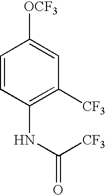

23.7 g (0.97 mol) of 4-trifluoromethoxy-2-trifluoromethylaniline are initially charged in 400 ml of methylene chloride and 58.6 g (0.58 mol) of triethylamine are added. At 20° C., 61 g (0.29 mol) of trifluoroacetic anhydride are then added, and the mixture is stirred at RT for 1 h. The organic phase is washed with water, dried over sodium sulphate and concentrated. The residue is distilled under reduced pressure.

Yield: 17.8 g (54% of theory), b.p._{0.5}: 68° C., GC: 93.7%.

### Example g

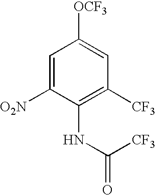

With cooling, 18 g (0.53 mol) of N-(4-trifluoromethoxy-2-trifluoromethyl)-trifluoroacetamide are dissolved in 50 ml of conc. sulphuric acid. With ice-cooling, a mixture of 50 ml of conc. sulphuric acid and 10 ml of conc. nitric acid is slowly added dropwise at 5° C. over a period of 2 h. The mixture is then poured onto ice and extracted with t-butyl methyl ether and the organic phase is washed with water and sodium chloride solution until neutral and dried over sodium sulphate. The organic phase is concentrated and dried under reduced pressure. Recrystallization from ethanol.

Yield: 13.2 g (65% of theory), GC: 96%.

### Example h

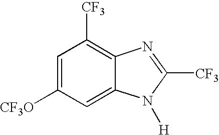

In an autoclave, 5 g (12.9 mmol) of N-(2-nitro-4-trifluoromethoxy-6-trifluoromethyl)-trifluoroacetamide are hydrogenated at a hydrogen pressure of 100 bar for 8 h using 1 g of Raney nickel in 50 ml of methanol. The Raney nickel is then filtered off and washed with methanol and the organic phase is concentrated. The residue is admixed with methylene chloride, washed twice with a little water and dried over sodium sulphate. The residue still contains N-(2-amino-4-trifluoromethoxy-6-trifluoromethyl)-trifluoroacetamide (GC). For this reason, the organic phase is treated with 25 ml of trifluoroacetic acid and 5 ml of trifluoroacetic anhydride and heated under reflux for 5 h. The mixture is cooled and adjusted to pH 8 using 20% strength sodium hydroxide solution. The mixture is extracted with methylene chloride and the organic phase is washed with water until neutral, dried over sodium sulphate and concentrated. The residue is dried under reduced pressure.

Yield: 4.1 g (94% of theory), GC: 85.6%.

Biological Examples

Cage Experiment on Coccidiosis/chicks

8- to 12- day-old male chicks (e.g. LSL Brinkschulte/Senden) which have been reared coccidia-free receive the compounds according to the invention (test substances) in the concentration indicated in ppm with the feed from 3 days before (day −3) infection (=a.i.) until 8 (9) days after infection (=p.i.). 3 animals are kept in each cage. One or more groups of this type are employed per dose. Infection is carried out by means of a stomach tube directly into the crop with approximately 100,000 sporulated oocysts of Eimeria acervulina and with approximately 30,000 oocysts each of E. maxima and 40,000 sporulated oocysts of E. tenella. These are highly virulent strains. The exact infection dose is adjusted so that, if possible, one of three experimentally infected untreated chicks dies due to the infection. For assessment of the efficacy, the following criteria are taken into account: weight increase from the start of the experiment to the end of the experiment, death rate due to infection, macroscopic assessment of the faeces with respect to diarrhoea and excretion of blood on days 5 and 7 p.i. (assessment 0 to 6), macroscopic assessment of the intestinal mucosa, in particular of the appendices (assessment 0 to 6) and the oocyst excretion as well as the proportion (in %) of the oocysts sporulating in the course of 24 hours. The number of oocysts in the faeces was determined with the aid of a McMaster counting chamber (see Engelbrecht and coworkers “Parasitologische Arbeitsmethoden in Medizin and Veterinärmedizin” [Parasitological Working Methods in Medicine and Veterinary Medicine], Akademie-Verlag, Berlin (1965)). The individual findings are related to the untreated non-infected control groups and a total score is calculated (cf. A. Haberkorn (1986), pp. 263 to 270 in Research in Avian Coccidiosis ed L. R. McDougald, L. P. Joyner, P. L. Long, Proceedings of the Georgia Coccidiosis Conference, Nov. 18-20, 1985, Athens/Ga. USA).

Experimental results with combinations according to the invention are shown by way of example in the following tables. The synergistic activity of the combinations in comparison with the individual components is particularly evident in the reduction of oocysts excretion but also with respect to the section findings, weight gain and better tolerability.

In the following tables, in the column “Treatment” the information means

n.inf.contr.=non-infected control group

inf.contr.=infected control group

1=benzimidazole example No.

In the column “ppm”, the concentration of the active compound employed in the feed is indicated in ppm.

In the column “mortality”, the percentage of the dead animals is indicated under % and the number of dead animals/animals employed in the experiment is indicated under n.

In the column “weight % of not inf. control”, the ratio of the weight of the treated animals to the weight of the non-infected control group is indicated.

In the columns “dropping scores”, “lesion score” and “oocyst control”, individual details of the action are given.

In the column “% efficacy”, the total score is assessed; 0% means no action, 100% means full action.

The results of the efficacy experiments with the compounds according to the invention are summarized in the following tables.

TABLE 1
Efficacy against Eimeria acervulina, Eimeria maxima
and Eimeria tenella

| | 50 | 25 | 10 | 7.5 | 5 | 2.5 | 1 |
|---|---|---|---|---|---|---|---|
| Comp. | ppm | ppm | ppm | ppm | ppm | ppm | ppm |
| Ex. 1 | NT | NT | 2 2 1 | 2 2 2 | 2 2 2 | 2 2 2 | 0 1 1 |
| Ex. 2 | 2 2 2 | 2 2 2 | 1 2 1 | 1 1 1 | 0 1 0 | 1 1 1 | 1 1 1 |
| Ex. 3 | NT | NT | 1 1 1 | 2 2 2 | 2 2 2 | 2 2 1 | 0 0 0 |

Assessment scheme:
2 = full action
1 = slight action
0 = inactive
D = death
NT = not tested

Assessment Scheme:

2=full action

1=slight action

0=inactive

D=death

NT=not tested

TABLE 2
Experimental infection with Eimeria acervulina, Eimeria maxima and Eimeria tenella
in chicks. Ex. 2 in combination with maduramicin (MAD)

| | | | | weight % | | | | | | | % |
|---|---|---|---|---|---|---|---|---|---|---|---|
| | | | | of not | drop- | | | | | | effi- |
| | | mortality | | inf. | ping | lesion | oocyst in % of inf. control | | | | cay |
| Treatment | ppm | % | n | control | scores | score | ac. | max. | ten. | tot. | tot. |
| n. inf. | 0 | 0 | 0/6 | 100 | 0 | 0 | 0 | 0 | 0 | 0 | 100 |
| contr. | | | | | | | | | | | |
| inf. | 0 | 0 | 0/6 | 46 | 6 | 6 | 100 | 100 | 100 | 100 | 0 |
| contr. | | | | | | | | | | | |
| MAD | 1 | 67 | 2/3 | 17 | 6 | 6 | >100 | >100 | 85 | >100 | 0 |
| MAD | 2 | 0 | 0/3 | 82 | 2 | 6 | >100 | >100 | >100 | >100 | 29 |
| MAD | 3 | 0 | 0/3 | 98 | 0 | 1 | >100 | >100 | 75 | >100 | 44 |
| Ex. 2 | 2.5 | 33 | 1/3 | 33 | 6 | 6 | >100 | >100 | >100 | >100 | 2 |
| Ex. 2 | 5 | 33 | 1/3 | 65 | 6 | 6 | 54 | 0 | 98 | 73 | 37 |
| Ex. 2 | 7.5 | 33 | 1/3 | 74 | 6 | 6 | 56 | 0 | 23 | 41 | 36 |
| Ex. 2 | 10 | 0 | 0/3 | 69 | 6 | 6 | 93 | >100 | 65 | 83 | 17 |
| Ex. 2 + | 10 + | 0 | 0/3 | 118 | 0 | 1 | 0 | 0 | 0 | 0 | 98 |
| MAD | 1 | | | | | | | | | | |
| Ex. 2 + | 7.5 + | 0 | 0/3 | 103 | 0 | 1 | 4 | 0 | 1 | 3 | 93 |
| MAD | 2 | | | | | | | | | | |
| Ex. 2 + | 10 + | 0 | 0/3 | 113 | 0 | 0 | 0 | 0 | 0 | 0 | 100 |
| MAD | 2 | | | | | | | | | | |
| Ex. 2 + | 2.5 + | 0 | 0/3 | 115 | 0 | 2 | 1 | 30 | 3 | 2 | 78 |
| MAD | 3 | | | | | | | | | | |
| Ex. 2 + | 5 + | 0 | 0/3 | 135 | 0 | 0 | 0 | 0 | 0 | 0 | 100 |
| MAD | 3 | | | | | | | | | | |
| Ex. 2 + | 7.5 + | 0 | 0/3 | 99 | 0 | 0 | 0 | 0 | 0 | 0 | 100 |
| MAD | 3 | | | | | | | | | | |
| Ex. 2 + | 10 + | 0 | 0/3 | 85 | 0 | 0 | 0 | 0 | 0 | 0 | 91 |
| MAD | 3 | | | | | | | | | | |

1. Benzimidazoles of the formula (I) 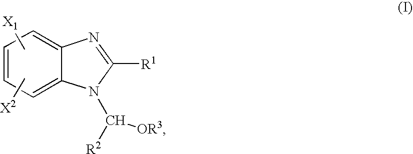
in which
R¹ represents fluoroalkyl,
R² represents hydrogen or alkyl,
R³ represents alkyl,
X¹ represents trifluoromethyl and
X² represents trifluoromethoxy.

2. Process for preparing compounds according to claim 1 of the formula (I) 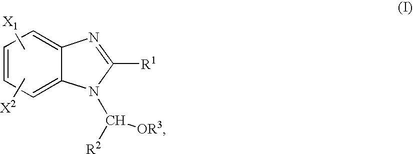
in which
R¹, R², R³, X¹ and X² have the meanings given in claim 1,
characterized in that
1H-benzimidazoles of the formula (II) 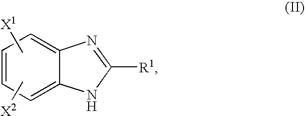
in which
R¹, X¹and X² have the meaning given in claim 1,
are reacted with an alkylating agent of the formula (III) 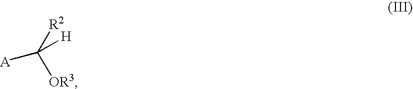
in which
A represents a suitable leaving group,
R² and ³ have the meaning given in claim 1,
if appropriate in the presence of diluents and/or reaction auxiliaries.

3. Compositions, characterized in that they comprise at least one compound of the formula (I) according to claim 1.

4. Compositions according to claim 3, characterized in that they comprise, as further active compound, a polyether antibiotic or a synthetic coccidiosis agent.

5. Compositions according to claim 4, characterized in that the polyether antibiotic or the synthetic coccidiosis agent is selected from the group consisting of: amprolium, robenidin, toltrazuril, monensin, salinomycin, maduramicin, lasalocid, narasin, semduramicin.

6. Use of compounds of the formula (I) according to claim 1 for controlling parasites.

7. Method for controlling parasites, characterized in that compounds of the formula (I) according to claim 1 are allowed to act on parasites.

8. Process for preparing antiparasitic compositions, characterized in that compounds of the formula (I) according to claim 1 are mixed with extenders and/or surfactants.

9. Use of compounds of the formula (I) according to claim 1 for preparing antiparasitic compositions.
